(19) United States
(12) Patent Application Publication
(10) Pub. No.: US 2025/0195862 A1
(43) Pub. Date: Jun. 19, 2025

(54) AUTOMATIC DETECTION OF DIRTY OR CONTAMINATED CONNECTORS IN A PATIENT MONITORING SYSTEM

(71) Applicant: Draeger Medical Systems, Inc., Andover, MA (US)

(72) Inventor: Clifford Risher-Kelly, Wells, ME (US)

(21) Appl. No.: 18/975,751

(22) Filed: Dec. 10, 2024

Related U.S. Application Data
(60) Provisional application No. 63/610,341, filed on Dec. 14, 2023.

(51) Int. Cl. A61M 39/16

(52) U.S. Cl. CPC A61M 39/16

(57) ABSTRACT

A medical system and method for the automatic detection of a dirty or contaminated connector port in a patient monitoring system. The system and method comprise one or more sensors affixed to a patient and configured to measure parameters of the patient and transmit the information to a patient monitor via a cable. The patient monitor may include a memory configured to store one or more software programs that are executed by one or processors. The system and method may be configured to execute one or more programs to receive the measured patient parameters, determine an indication of a disconnected cable, determine if the connection interface is contaminated based on a detection algorithm, and provide an alert on a display if the detection algorithm indicates that the interface is contaminated.

### CROSS-REFERENCE TO RELATED APPLICATION(S)

The present application claims priority to U.S. Provisional Application No. 63/610,341, filed on Dec. 14, 2023, titled, “Automatic Detection of Dirty or Contaminated Connectors in a Patient Monitoring System,” which is hereby incorporated by reference in its entirety.

### TECHNICAL FIELD

In general, the present disclosure relates to the field of patient monitoring systems. More specifically, the present disclosure relates to the automatic detection of dirty and/or contaminated connectors, which are typically connected to patient monitors via cables, and disconnected cables.

### BACKGROUND

In general, patient monitoring systems are often comprised of a monitoring device connected to a patient via one or more cables. These cables typically include sensors that measure, monitor, or obtain one or more parameters of the patient. These parameters are then displayed on a display of the patient monitor. These patient monitors are used by healthcare facilities to monitor and display information about a patient, such as identifying data, vital signs, medications that have been administered to the patient, and may be connected to patient record systems and able to display patient history information. Patient monitors may be portable devices that travel with the patient in order to provide continuous monitoring during care.

Additionally, during the course of providing treatment to patients, medical practitioners typically connect at least one type of sensor to a patient to sense, derive, or otherwise monitor at least one biometric parameter. While some sensors and connectors may be one-time-use components, other components are reusable. The type of connection between the sensor and the patient monitor may be a wired electrical connection, a wired optical connection, or a wireless connection, to list a few examples. Accordingly, these reusable components often need to be cleaned and/or sterilized between users. In recent years, cleaning agents have become both more efficient and more aggressive. Likewise, the amount of time that medical equipment is exposed to these cleaning agents, and remains in contact with these agents, has also increased. This has resulted in residues, or other contaminants, remaining on the connectors after the cleaning and sterilization processes in some cases.

### SUMMARY

Some patient monitors are able to detect the connection and removal of cables. Likewise, some cables can self-identify the types of cables and/or sensors such that the patient monitor is informed of the type of cable being connected to the patient monitor. The present system and method provide a way to determine if a connector is contaminated and/or dirty, which will help reduce and/or eliminate missed or incorrect alarms due to potentially corrupted signal information. In one example, a dirty connection could cause an incorrect signal to be received by the patient monitor. This, in turn, could then cause the patient monitor to fail to recognize a dangerous condition for which an alarm should have been generated. Alternatively, the signal may be corrupted such that a false alarm is generated. Thus, it would be beneficial to be able to identify dirty or contaminated connections prior to the cables and associated sensors being used to measure the parameters of the patient. This will reduce the likelihood of false alarms, missed alarms, and generally provide better outcomes for patients.

In general, the present system may include a patient monitoring system, comprising: one or more sensors attached to a patient and configured to measure parameters of the patient, the one or more sensors connected to the patient monitor via a cable, a memory configured to store one or more software programs that are executed by one or more processors of the patient monitor. Additionally, the present system may further include one or more processors that are communicatively connected to the memory and configured to execute the one or more programs to: receive the measured patient parameters; determine an indication of a disconnected cable; determine if a connection interface is contaminated based on a detection algorithm; and provide an alert on a display if the detection algorithm indicates that the interface is contaminated.

The present system may further include a patient monitor which comprises a plurality of connection interfaces and one or more processors that are configured to identify which connection interface of the plurality of connection interfaces is contaminated. The present system may further include in some embodiments a detection algorithm that is a lead-off detection algorithm that is executed upon a determination of a disconnected cable and wherein the lead-off detection algorithm measures the impedance of the pins of the connection interface to determine if the impedance exceeds a predefined threshold. In one embodiment, the predefined threshold is 100 MegaOhms. The present system may further include one or more processors are configured to prevent data transmission via the interface connection in response to a determination that the interface connection is contaminated and provide an alert that includes a user-selectable interface that is configured to require a user to acknowledge the alert prior to enabling data transmission via the connection interface. Additionally, The present system may further include an alert which enables the user-selectable inputs to override the prevention of the data transmission. Additionally, the one or more sensors measure at least one of electrocardiogram (ECG), non-invasive peripheral oxygen saturation (SpO2), non-invasive blood pressure (NIBP), temperature, neuromuscular transmission (NMT), and end-tidal carbon dioxide (ETCO2), to list a few examples.

In another embodiment, a method for automatic detection of contaminated connectors may include one or more of the steps of attaching one or more sensors to a patient that are configured to measure parameters of the patient, the one or more sensors connected to a patient monitor via a cable, receiving patient parameters measured by the one or more sensors attached to the patient, determining if the disconnected cable is disconnected, determining if a connection interface between the cable and patient monitor is contaminated based on a detection algorithm, and displaying an alert on a display, if the detection algorithm indicates that the interface is contaminated.

The method may further include the patient monitor comprising a plurality of connection interfaces and one or more processors being configured to identify which connection interface of the plurality of connection interfaces is contaminated. The method may further comprise in some embodiments performing a lead-off detection algorithm upon the determination of the disconnected cable. The method may further comprise a lead-off detection algorithm that measures an impedance of the pins of the connection interface to determine if the impedance exceeds a predefined threshold. In one embodiment, the predefined threshold is 100 MegaOhms.

The method may further comprise preventing data transmission via the interface connection in response to the determination that the interface connection is contaminated. The method may further comprise displaying an alert that includes a user-selectable interface that is configured to require a user to acknowledge the alert prior to enabling data transmission via the connection interface. The method may further comprise displaying user-selectable inputs that override the prevention of the data transmission. In one embodiment, the one or more sensors measure at least one of ECG, SpO2,NIBP, temperature, NMT, and ETCO2.

### BRIEF DESCRIPTION OF THE DRAWINGS

In the drawings, like reference numbers generally indicate identical, functionally similar, and/or structurally similar elements.

FIG. 1 is a schematic diagram of an example of a system capable of executing a customizable physiological measurement schedule for measuring physiological parameters according to an embodiment of the present disclosure;

FIG. 2 is a schematic diagram of an example of a physiological monitoring device capable of executing a customizable physiological measurement schedule for measuring physiological parameters according to an embodiment of the present disclosure;

FIG. 3 is a schematic diagram of an example of a system including a server/central computer according to an embodiment of the present disclosure;

FIG. 4 is a schematic diagram of an example of a server/central computer according to an embodiment of the present disclosure;

FIG. 5 is a schematic diagram illustrating one example of a lead-off detection system in accordance with the present disclosure;

FIG. 6 is a flow diagram illustrating steps performed by a process of the patient monitor in accordance with the present disclosure; and

FIG. 7 is a schematic diagram illustrating one example of contamination detection in accordance with the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

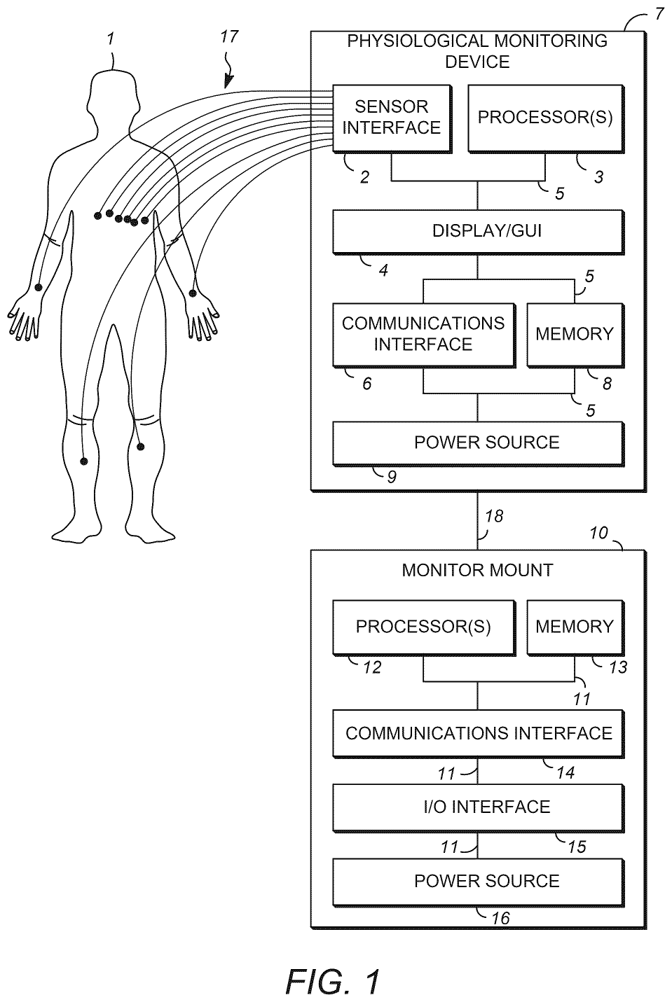

FIG. 1 is a schematic diagram of an example of a system capable of executing a customizable physiological measurement schedule for measuring physiological parameters according to an embodiment of the present disclosure. As shown in FIG. 1, the system includes an electronic device such as a physiological monitoring device 7 (often referred to as a patient monitor) capable of receiving physiological data from various sensors 17 connected to a patient 1, and a monitor mount 10, to which the physiological monitoring device 7 is removably mounted or docked.

In general, it is contemplated by the present disclosure that the physiological monitoring device 7 and the monitor mount 10 include electronic components or electronic computing devices operable to receive, transmit, process, store, and/or manage patient data and information associated with performing the functions of the system, which encompasses any suitable processing device adapted to perform computing tasks consistent with the execution of computer-readable instructions stored in a memory or a computer-readable recording medium.

Further, any, all, or some of the computing devices in the physiological monitoring device 7 and the monitor mount 10 may be adapted to execute any operating system, including Linux, UNIX, Windows Server, etc., as well as virtual machines adapted to virtualize execution of a particular operating system, including customized and proprietary operating systems. The physiological monitoring device 7 and the monitor mount 10 are further equipped with components to facilitate communication with other computing devices over one or more network connections, which may include connections to local and wide area networks, wireless and wired networks, public and private networks, and any other communication network enabling communication in the system.

As shown in FIG. 1, the physiological monitoring device 7 is, for example, a portable or stationary patient monitor implemented to monitor various physiological parameters of the patient 1 via the sensors 17. The physiological monitoring device 7 includes a sensor interface 2, one or more processors 3, a display 4 including a graphical user interface (GUI), a communications interface 6, a memory 8, and a power source 9. The sensor interface 2 can be implemented in software or hardware or a combination thereof and used to connect via wired and/or wireless connections to one or more physiological sensors 17 for gathering physiological data from the patient 1.

The data signals from the sensors 17 include, for example, data related to an electrocardiogram (ECG), non-invasive peripheral oxygen saturation (SpO2), non-invasive blood pressure (NIBP), temperature, and/or end tidal carbon dioxide (etCO2), apnea detection, neuromuscular transmission (NMT), and cardiac output (CO), or other similar physiological data that can be measured discretely or continuously. The one or more processors 3 are used for controlling the general operations of the physiological monitoring device 7.

The display 4 is for displaying various patient data, measurement schedules, and hospital or patient care information and for allowing communication between a user and the physiological monitoring device 7. The display 4 includes, but is not limited to, a keyboard, a liquid crystal display (LCD), thin film transistor (TFT), light-emitting diode (LED), high definition (HD), or other similar GUI with touch screen capabilities. The patient information displayed can, for example, relate to the measured physiological parameters of the patient 1 (e.g., blood pressure, heart-related information, pulse oximetry, respiration information, etc.) as well as information related to a customizable measurement schedule for taking the physiological parameters of the patient 1.

The communications interface 6 allows the physiological monitoring device 7 to directly or indirectly (via, for example, the monitor mount 10) communicate with one or more computing networks and devices. The communications interface 6 can include various network cards, interfaces, or circuitry to enable wired and wireless communications with such computing networks and devices. The communications interface 6 can also be used to implement, for example, a Bluetooth connection, a cellular network connection, and/or a Wi-Fi connection. Other wireless communication connections implemented using the communications interface 6 include wireless connections that operate in accordance with, but are not limited to, IEEE802.11 protocol, a Radio Frequency For Consumer Electronics (RF4CE) protocol, ZigBee protocol, and/or IEEE802.15.4 protocol.

Additionally, the communications interface 6 can enable direct (i.e., device-to-device) communications (e.g., messaging, signal exchange, etc.) such as from the monitor mount 10 to the physiological monitoring device 7 using, for example, a USB connection. The communications interface 6 can also enable direct device-to-device connection to other devices such as to a tablet, PC, or similar electronic device, or to an external storage device or memory.

The memory 8 can be used to store any type of instructions, patient data, and measurement schedules associated with algorithms, processes, or operations for controlling the general functions and operations of the physiological monitoring device 7.

The power source 9 can include a self-contained power source such as a battery pack and/or include an interface to be powered through an electrical outlet (either directly or by way of the monitor mount 10). The power source 9 can also be a rechargeable battery that can be detached allowing for replacement. In the case of a rechargeable battery, a small built-in backup battery (or super capacitor) can be provided for continuous power to be provided to the physiological monitoring device 7 during battery replacement. Communication between the components of the physiological monitoring device 7 (e.g., 2, 3, 4, 6, 8, and 9) is established using an internal bus 5.

As shown in FIG. 1, the physiological monitoring device 7 is connected to the monitor mount 10 via a connection 18 that establishes a communication connection between, for example, the respective communications interfaces 6, 14 of the devices 7, 10. The connection 18 enables the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a user when desired. The connection 18 may include, but is not limited to, a universal serial bus (USB) connection, parallel connection, a serial connection, coaxial connection, a High-Definition Multimedia Interface (HDMI) connection, or other similar connection known in the art connecting to electronic devices. Additionally, the connection may include optical communications interfaces and/or high-speed wireless communication interfaces.

The monitor mount 10 includes one or more processors 12, a memory 13, a communications interface 14, an I/O interface 15, and a power source 16. The one or more processors 12 are used for controlling the general operations of the monitor mount 10. The memory 13 can be used to store any type of instructions associated with algorithms, processes, or operations for controlling the general functions and operations of the monitor mount 10. The one or more processors (or processing unit) 12 are used for controlling the general operations of the monitor mount 10. The one or more processors may be any suitable processor-based resource known to the art. They may be, but are not limited to, a central processing unit (“CPU”), a hardware microprocessor, a multi-core processor, a single core processor, a field programmable gate array (“FPGA”), a controller, a microcontroller, an application specific integrated circuit (“ASIC”), a digital signal processor (“DSP”), or other similar processing device capable of executing any type of instructions, algorithms, or software. In some embodiments, the one or more processors 12 may comprise a processor chipset including, for example and without limitation, one or more co-processors.

The communications interface 14 allows the monitor mount 10 to communicate with one or more computing networks and devices (e.g., the physiological monitoring device 7). The communications interface 14 can include various network cards, interfaces, or circuitry to enable wired and wireless communications with such computing networks and devices. The communications interface 14 can also be used to implement, for example, a Bluetooth connection, a cellular network connection, and a Wi-Fi connection. Other wireless communication connections implemented using the communications interface 14 include wireless connections that operate in accordance with, but are not limited to, IEEE802.11 protocol, a Radio Frequency For Consumer Electronics (RF4CE) protocol, ZigBee protocol, and/or IEEE802.15.4 protocol.

The communications interface 14 can also enable direct (i.e., device-to-device) communications (e.g., messaging, signal exchange, etc.) such as from the monitor mount 10 to the physiological monitoring device 7 using, for example, a USB connection, coaxial connection, or other similar electrical connection. The communications interface 14 can enable direct (i.e., device-to-device) to other devices such as to a tablet, PC, or similar electronic device, or to an external storage device or memory.

The input/output (I/O) interface 15 can be an interface for enabling the transfer of information between monitor mount 10, one or more physiological monitoring devices 7, and external devices such as peripherals connected to the monitor mount 10 that need special communication links for interfacing with the one or more processors 12. The I/O interface 15 can be implemented to accommodate various connections to the monitor mount 10 that include, but are not limited to, a universal serial bus (USB) connection, parallel connection, a serial connection, a coaxial connection, a High-Definition Multimedia Interface (HDMI) connection, or other known connection in the art connecting to external devices.

The power source 16 can include a self-contained power source such as a battery pack and/or include an interface to be powered through an electrical outlet. The power source 16 can also be a rechargeable battery that can be detached allowing for replacement. Communication between the components of the monitor mount 10 (e.g., 12, 13, 14, 15, and 16) is established using an internal bus 11.

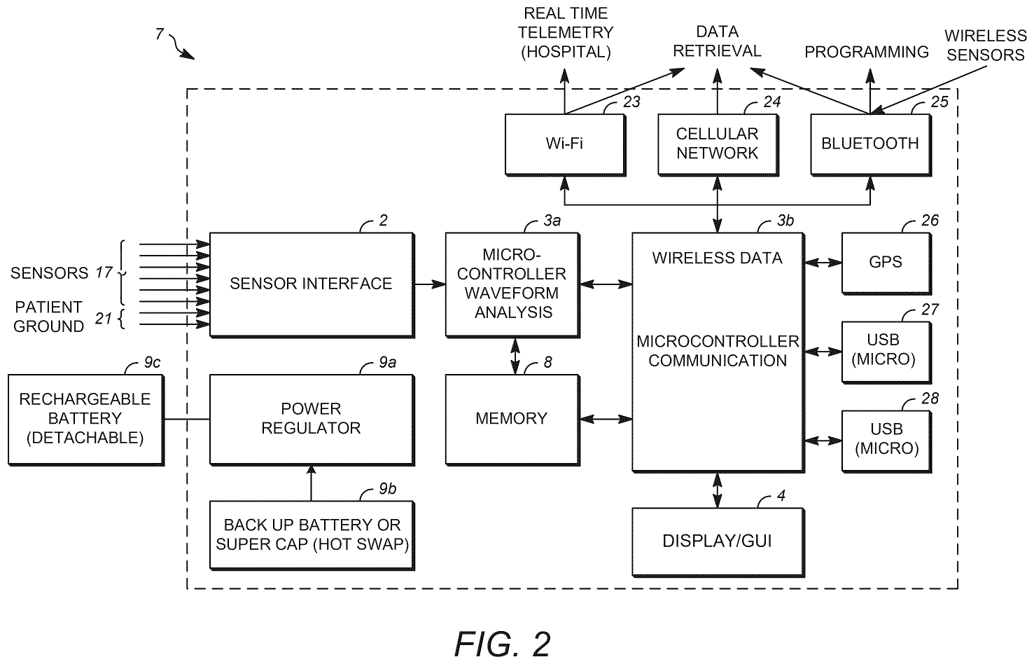

FIG. 2 is a schematic diagram of an example of a physiological monitoring device 7 capable of executing a customizable physiological measurement schedule for measuring physiological parameters according to an embodiment of the present disclosure.

As shown in FIG. 2, the physiological monitoring device 7 is attached to several different types of sensors 17 (including electrodes or other similar devices) known in the art for gathering physiological data related to the patient 1 (e.g., as shown on the left side of FIG. 1). The sensors 17 are communicatively coupled to physiological monitoring device 7 by, for example, a wired connection input to the sensor interface 2. It is contemplated by the disclosure that the physiological monitoring device 7 can also be connected to other wireless sensors using the communication interface 6, which includes circuitry for receiving data from and sending data to one or more devices using, for example, a Bluetooth connection 25. The communications interface 6 shown in FIG. 1 is represented in FIG. 2 by the combination of microcontroller 3b and elements 23-28.

The data signals from the sensors 17 received by the physiological monitoring device 7 include data related to, for example, an ECG, SpO2, NIBP, temperature, and/or ETCO2. The data signals received from an ECG sensor and the SpO2 sensor can be analog signals. The data signals for the ECG and the SpO2 are input to the sensor interface 2, which can include an ECG data acquisition circuit and a SpO2 data acquisition circuit. Both the ECG data acquisition circuit and the SpO2 data acquisition circuit include amplifying and filtering circuity as well as analog-to-digital (A/D) circuity that convert the analog signal to a digital signal using amplification, filtering, and A/D conversion methods known in the art.

As another example, the data signals related to non-invasive blood pressure (NIBP), temperature, and end-tidal carbon dioxide (ETCO2) can be received from sensors 17 to the sensor interface 2, which can include a physiological parameter interface such as serial interface circuitry for receiving and processing the data signals related to NIBP, temperature, and etCO2. The ECG data acquisition circuit, an SpO2 data acquisition circuit, and a physiological parameter interface are described as part of the sensor interface 2. However, it is contemplated by the present disclosure that the ECG data acquisition circuit, the SpO2 data acquisition circuit, and the physiological parameter interface can be implemented as circuits separate from the sensor interface 2.

The processing performed by the ECG data acquisition circuit, the SpO2 data acquisition circuit, and external physiological parameter interface produces digital data waveforms that are analyzed by the microcontroller 3a. The processors 3 shown in FIG. 1 are represented in FIG. 2 as microcontrollers 3a and 3b. The microcontroller 3a, for example, analyzes the digital waveforms to identify certain digital waveform characteristics and threshold levels indicative of conditions (abnormal and normal) of the patient 1 using methods known in the art. The microcontroller 3a includes a memory or uses the memory 8.

The memory 8 stores software or algorithms with executable instructions and the microcontroller 3a can execute a set of instructions of the software or algorithms in association with executing different operations and functions of the physiological monitoring device 7 such as analyzing the digital data waveforms related to the data signals from the sensors 17. The results of the operations performed by the microcontroller 3a are passed to the microcontroller 3b. The microcontroller 3b includes a memory or uses the memory 8.

As noted above, in FIG. 2, the communication interface 6 shown in FIG. 1 is represented by the combination of microcontroller 3b and elements 23-28. For example, the microcontroller 3b includes communication interface circuitry for establishing communication connections with various devices and networks using both wired and wireless connections, and transmitting physiological data, patient and transport information (e.g., transport times and patient location information), results of the analysis by the microcontroller 3a, and alerts and/or alarms to the patient 1, clinicians and/or caregivers. The memory 8 stores software or algorithms with executable instructions and the microcontroller 3b can execute a set of instructions of the software or algorithms in association with establishing the communication connections.

As shown in FIG. 2, wireless communication connections established by the communication interface circuity of microcontroller 3b include a Bluetooth connection 25, a cellular network connection 24, and a Wi-Fi connection 23. The wireless communication connections can allow, for example, patient and hospital information, alerts, and physiological data to be transmitted in real-time within a hospital wireless communications network (e.g., Wi-Fi) as well as allow for patient and hospital information, alerts, and physiological data to be transmitted in real-time to other devices (e.g., Bluetooth 25 and/or cellular networks 24).

It is also contemplated by the present disclosure that the communication connections established by the microcontroller 3b enable communications over other types of wireless networks using alternate hospital wireless communications such as wireless medical telemetry service (WMTS), which can operate at specified frequencies (e.g., 1.4 GHZ). Other wireless communication connections can include wireless connections that operate in accordance with, but are not limited to, IEEE802.11 protocol, a Radio Frequency For Consumer Electronics (RF4CE) protocol, ZigBee protocol, and/or IEEE802.15.4 protocol.

The Bluetooth connection 25 can also be used to provide the transfer of data to a nearby device (e.g., tablet) for review of data and/or changing of operational settings of physiological monitoring device 7. The microcontroller 3b of the physiological monitoring device 7 provides a communication connection by direct wired (e.g., hard-wired) connections for transferring data using, for example, a USB connection 27 to a tablet, PC, or similar electronic device; or using, for example, a USB connection 28 to an external storage device or memory. Additionally, the microcontroller 3b includes a connection to a display 4 including a GUI for displaying patient information, physiological data or measured data, measurement schedules, alerts or alarms for the patient, clinicians and/or caregiver's information. Although the physiological monitoring device 7 is described in FIG. 1 as having two microcontrollers 3a and 3b, it is contemplated by the disclosure of the present application that one microcontroller can be implemented to perform the functions of the two microcontrollers 3a and 3b.

The display 4 includes, for example, a liquid crystal display (LCD), thin film transistor (TFT), light-emitting diode (LED), high definition (HD), or other similar GUI with touch screen capabilities. The display 4 also includes a GUI that provides a means for inputting instructions or information directly to the physiological monitoring device 7. As shown in FIG. 2, the physiological monitoring device 7 includes a global positioning system (GPS) or other location data system 26 that can be connected to the communication interface circuity of microcontroller 3b so that the physiological monitoring device can transmit to the clinician, caregiver, or other devices the location of the patient 1 at all times including the location of the patient 1. Additionally, the location of the patient 1 can be used by the microcontroller 3b to determine an estimated time of arrival of the patient 1.

For example, location data provided by the location data system 26, which may include information on a floor level, can be compared to stored information related to a hospital layout or a hospital map as well as information related to a patient's scheduled care (e.g., treatment or procedure scheduled for the patient 1 in a patient care area within the hospital). Based on the comparison results, the microcontroller 3b can determine the estimated time of arrival of the patient 1 to the patient care area within the hospital. The estimated time of arrival can be transmitted by the communication interface circuity of microcontroller 3b to, for example, the hospital wireless communications system.

Additionally, if it is determined by the microcontroller 3b that the patient 1 is not within the vicinity of the hospital wireless communications system (e.g., based on input from the location data system 26), the pertinent physiological data can be recorded and stored in the memory 8. Additionally, if the Bluetooth connection 25 or WIFI connection 23 are not available (e.g., out of transmission range or not operable), then the microcontroller can store the physiological data in the memory 8 for later transmission when the Bluetooth connection or WIFI connection becomes available.

The power source 9 shown in FIG. 1 is represented by elements 9a-9c in FIG. 2. As shown in FIG. 2, the power can be supplied using a rechargeable battery 9c that can be detached allowing for replacement. The rechargeable battery 9c is, for example, a rechargeable lithium-ion battery. Additionally, a small built-in backup battery 9b (or supercapacitor) is provided for continuous power to the physiological monitoring device 7 during battery replacement. A power regulator or regulation circuit 9a is provided between the rechargeable battery 9c and small backup battery 9b to control which battery provides power to the physiological monitoring device 7. The physiological monitoring device 7 also includes a patient ground connection 21. The patient ground connection 21 can be used as a ground for single ended unipolar input amplifiers (e.g., precordial leads), or as a ground for bipolar input amplifiers (e.g., limb leads). It is also contemplated by the present disclosure that the power regulator 9a can include a self-contained power source such as a battery pack and/or include an interface to be powered through an electrical outlet (either directly or by way of the monitor mount 10). Additionally, or alternatively, the patient ground connection 21 could be a common mode voltage, which may be referred to as a Wilson Point or Wilson Central Terminal (WCT. The WCT is typically obtained by averaging the three active limb electrode voltages measured with respect to a return ground electrode. Communication between the components of the physiological monitoring device 7 can be established using an internal bus similar to the internal bus 5 discussed with reference to FIG. 1.

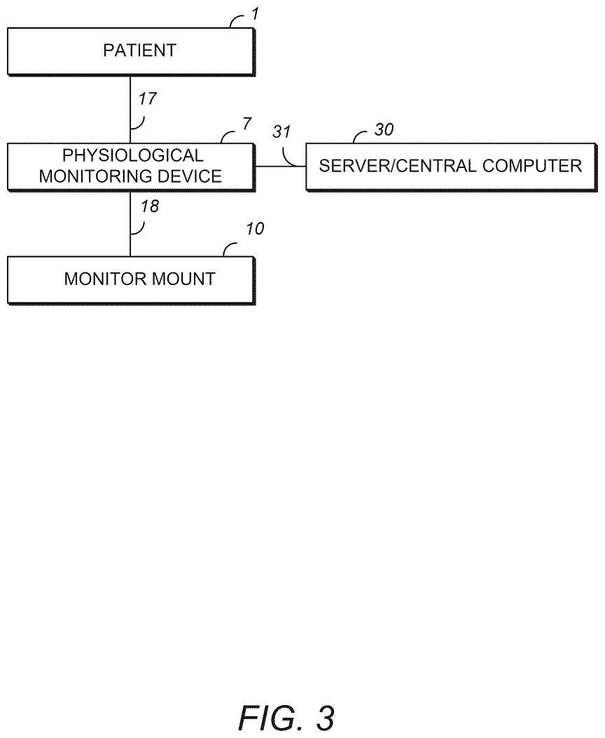

FIG. 3 is a schematic diagram of an example of a system including a server/central computer according to an embodiment of the present disclosure. FIG. 3 includes the patient 1, the physiological monitoring device 7, and the monitor mount 10 already discussed with reference to FIGS. 1 and 2. However, FIG. 3 also includes the addition of a server or central computer 30. As shown in FIG. 3, the physiological monitoring device 7 receives physiological data from various sensors 17 connected to the patient 1, and the physiological monitoring device 7 is removably mounted or docked to the monitor mount 10. The physiological monitoring device 7 is connected to the monitor mount 10 via the connection 18 that establishes a communication connection between, for example, the respective communications interfaces 6, 14 of the devices 7, 10. The connection 18 enables the monitor mount 10 to detachably secure the physiological monitoring device 7 to the monitor mount 10.

The connection 18 may include, but is not limited to, a universal serial bus (USB) connection, parallel connection, a serial connection, coaxial connection, a High-Definition Multimedia Interface (HDMI) connection, or other similar connection known in the art connecting to electronic devices. The physiological monitoring device 7 can also be connected to a server/central computer 30 via a wired or wireless connection 31 using the communication interface circuity of the communications interface 6 of the physiological monitoring device 7 described with reference to FIGS. 1 and 2. The server/central computer 30 can be located in or outside the hospital environment. For example, the server/central computer 30 can be located at a nurse station or other similar location within the hospital.

In one embodiment, the physiological monitoring device 7 may transmit, via the connection 31, physiological data collected by the sensors and/or other patient information (e.g., measurement schedules, patient location information, alert/alarm information) to the server/central computer 30 for storage and data processing. For example, upon the NIBP measurements with variable intervals configured by users on the monitoring device 7, the NIBP data processed by the monitoring device 7 along with related information may be transmitted and stored in the server/central computer 30.

In another embodiment, the server/central computer 30 may transmit control signals, via the connection 31, to control the functions of the monitoring device 7 and the sensors that are connected to the device. As such, users are allowed to control the physiological measurements performed by the sensors or configure the measurement settings, via the user interface of the server/central computer 30. For example, the server/central computer 30 may allow users to configure NIBP measurements (e.g., customize measurement intervals and/or frequencies) via the user interface of the server/central computer 30 without being in front of the monitoring device 7.

Optionally or additionally, the server/central computer 30 may store the patient's physiological measurements and algorithms to provide recommended measurement configurations to users based on one or more of the patient's physiological parameters, medical history, and care area where the patient is currently located. For example, based on the patient's NIBP trends in a pre-determined time, the patient's medical history and/or the care area where the patient is located, the algorithms in the server/central computer 30 may provide recommended measurement configurations in adjusting NIBP measurement intervals and/or frequencies.

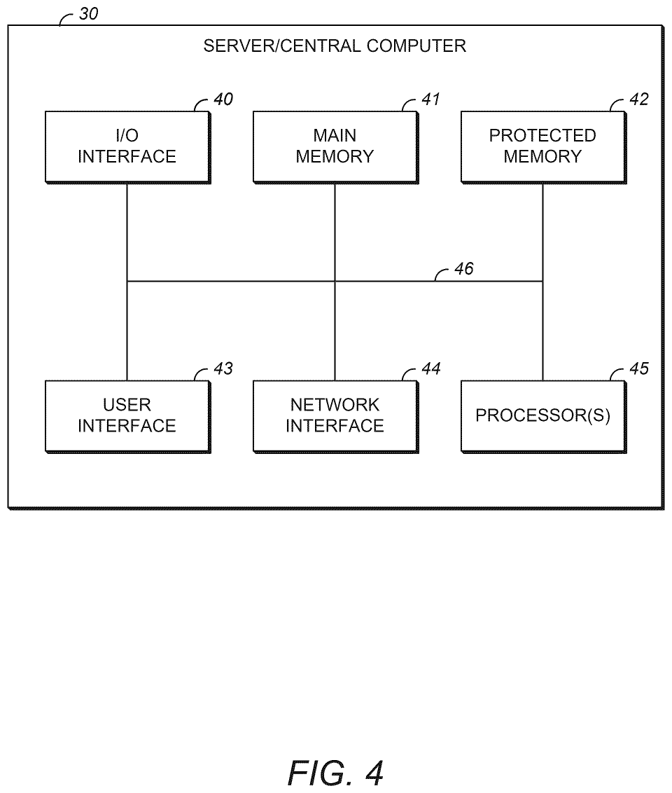

FIG. 4 is a schematic diagram of an example of a server/central computer according to an embodiment of the present disclosure. As shown in FIG. 4, the exemplary server/central computer 30 includes an I/O interface 40, a main memory 41, a protected memory 42, a user interface 43, a network interface 44, and one or more processors 45.

The I/O interface 40 can be implemented to accommodate various connections to the server/central computer 30 that include, but are not limited to, a universal serial bus (USB) connection, parallel connection, a serial connection, coaxial connection, a High-Definition Multimedia Interface (HDMI) connection, or other known connection in the art connecting to external devices. The I/O interface 40 can be an interface for enabling the transfer of information between server/central computer 30, one or more physiological monitoring devices 7, and external devices such as peripherals connected to the server/central computer 30 that need special communication links for interfacing with the one or more processors 45.

The main memory 41 can be used to store any type of instructions associated with algorithms, processes, or operations for controlling the general functions of the server/central computer 30 as well as any operating system such as Linux, UNIX, Windows Server, or other customized and proprietary operating systems.

The protected memory 42 is, for example, a processor reserved memory of dynamic random-access memory (DRAM) or other reserved memory module or secure memory location for storing more critical information such as confidential or proprietary patient information.

The user interface 43 is implemented for allowing communication between a user and the server/central computer 30. The user interface 43 includes, but is not limited to, a mouse, a keyboard, a liquid crystal display (LCD), thin film transistor (TFT), light-emitting diode (LED), high definition (HD) or other similar display device with touch screen capabilities. The network interface 44 is a software and/or hardware interface implemented to establish a connection between the server/central computer 30 and one or more physiological monitoring devices or other servers/central computer inside and outside the patient care or hospital environment.

It is contemplated by the present disclosure that network interface 44 includes software and/or hardware interface circuitry for establishing communication connections with the rest of the system using both wired and wireless connections for establishing connections to, for example, a local area networks (LANs), wide area networks (WANs), metropolitan area networks (MANs) personal area networks (PANs), and wireless local area networks (WLANs), system area networks (SANs), and other similar networks.

The one or more processors 45 are used for controlling the general operations of the server/central computer 30. Communication between the components of the server/central computer 30 (e.g., 40-44) is established using an internal bus 46.

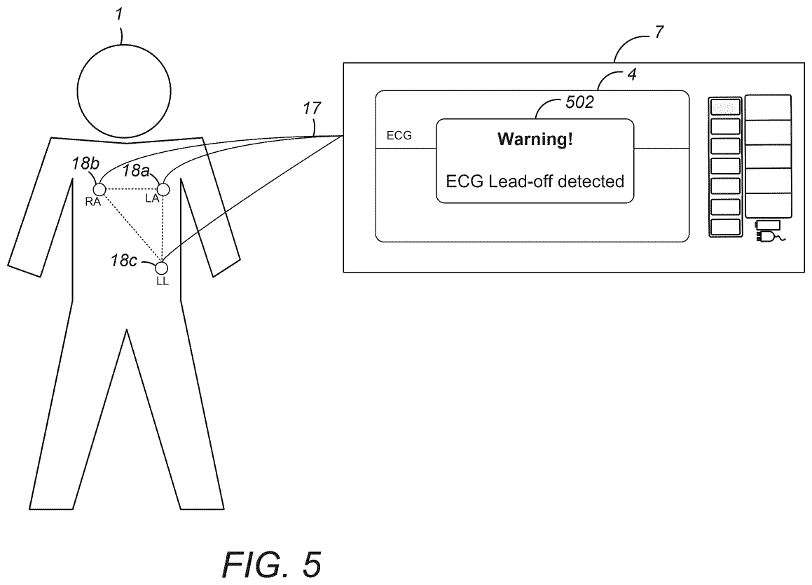

FIG. 5 is a schematic diagram illustrating one example of lead-off detection implemented by a patient monitor 7 in accordance with the present disclosure.

In the illustrated embodiment, the patient monitoring device 7 obtains ECG signals of the patient 1 via multiple electrical leads 18a, 18b, 18c, which are typically placed on the chest by the left arm (“LA”; 18a), on the chest by the right arm (“RA”; 18b). Lastly, a third lead (“LL;” 18c) is usually placed on the leg or lower abdomen and provides a “ground” (or common mode voltage, as detailed above) reference for the other signals. The location of these leads creates an Einthoven's Triangle with the heart at the center, which then enables electrical activity of the patient's heart may be monitored by the patient monitor 7. This illustrated embodiment is a 3-lead configuration. However, configurations implementing 5, 8, 10, and/or 12 lead configurations may also be used by medical professionals to obtain additional ECG signals (see, for example, FIG. 1, which illustrates a 10-lead configuration).

The patient monitoring device 7 may implement “lead-off” detection. That is, the patient monitoring device 7 may implement one or more algorithms that are able to detect if the electrical leads 18a, 18b, and/or 18c are not attached to the patient's body. One typical method for lead-off detection is based on a measured resistance. When one or more leads are “off,” then the measured resistance between LA and RA leads becomes extremely high, which is indicative of a lead-off scenario. Accordingly, in response to the measured resistance exceeding a threshold, a message 502 could be displayed on the display 4 of the patient monitor 7 alerting the user to disconnected or possibly non-functioning lead.

Some newer patient monitoring systems implement optical detection of the cables being connected and may also use, for example, an EEPROM (Electrically Erasable Programmable Read-Only Memory) that can specify the type of cable (e.g., ETCO2, SPO2, ECG, NIBP, etc.) connected the patient monitor 7. Accordingly, the presence of these cables may be checked optically to determine if the plug is not inserted into the patient monitor 7. Upon determining that a cable is disconnected from the patient monitor 7, the patient monitor 7 may implement a routine to further check whether the connector of the cable has been contaminated. This may be accomplished by implementing a lead-off detection algorithm, even though the patient monitor has already detected that the cable has been disconnected.

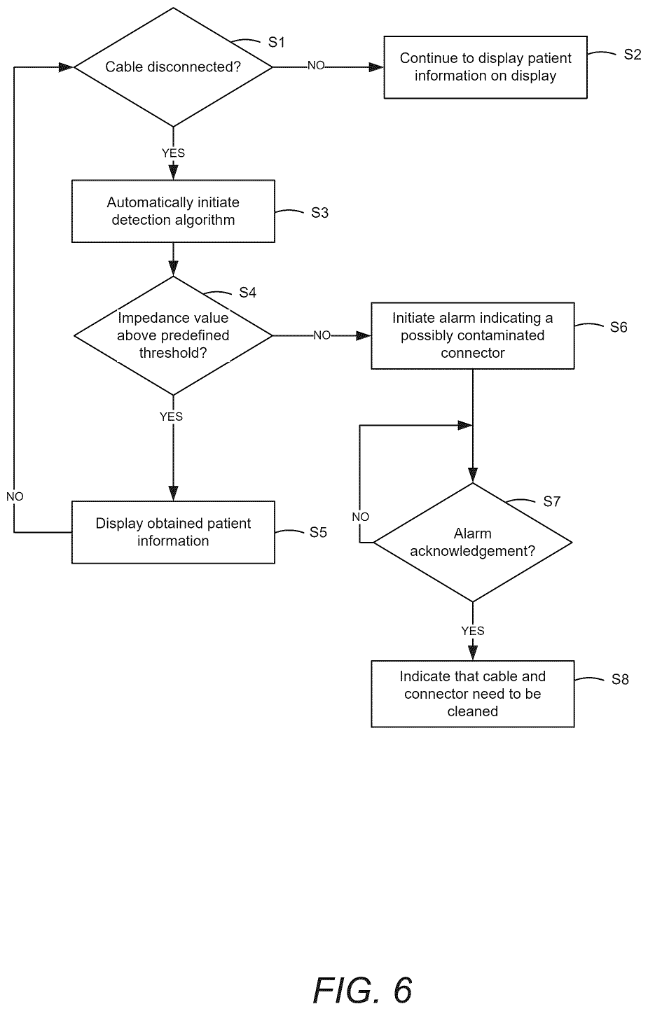

FIG. 6 illustrates one example of the software routine, which determines when a cable is connected, and then executes an algorithm to identify potentially contaminated connections.

Step S1 determines if a cable, which was previously connected to the patient monitor 7, is disconnected. Typically, cables and connectors are connected, cleaned or disinfected, and then reconnected to the patient monitor between patient use. That is, after a first patient uses a plurality of cables and the patient monitor, the room and all the equipment are cleaned and disinfected. Later, a different patient may be admitted (or possibly the same patient readmitted) and uses the same equipment as the first patient. Alternatively, the cables and associated sensors may be one-time uses cables, but the patient monitor is not. Thus, while cleaning and disinfecting the patient monitor the ports on the monitor may be contaminated.

As illustrated in the figures, as long as the cable remains connected to the patient monitor 7 (e.g., while in use from a patient), then the patient information will continue to be displayed on the patient monitor 7 in step S2. If, however, the cable is disconnected (e.g., between patients or to switch out and/or swap cables), then the patient monitor 7 automatically initiates the detection algorithm in step S3. In one example, the detection algorithm S3 is a repurposed lead-off detection algorithm, which measures an impedance of the pins of the port, from which the cable was just connected. In step S4, if the measured impedance value is above the predefined threshold, then the patient monitor 7 displays the obtained patient information in step S5. Then, as illustrated, the software routine then returns to step S1 to determine if the cable has been connected. In one embodiment, the predefined threshold value is 100 Megaohms. Alternatively, measuring less than the predefined threshold determined indicates that the connection is possibly contaminated and connector should be cleaned before any measured and/or obtained patient information is displayed as it may not be clinically accurate (e.g., the patient information is not correct, accurate, and possibly corrupted due to contamination.

Returning to Step S4, if the impedance value does not exceed the threshold, then the patient monitor 7 initiates an alarm indicating a possibly contaminated connector in step S6. The alarm could be visual and/or audible. Additionally, the patient monitor 7 may further identify the cable and/or input port, which is contaminated. Identifying the port enables users to quickly identify the problematic connection and cable. Likewise, in some scenarios, the cable and/or port may be prevented from operating until the measured impedance is above the predefined threshold (e.g., step S4).

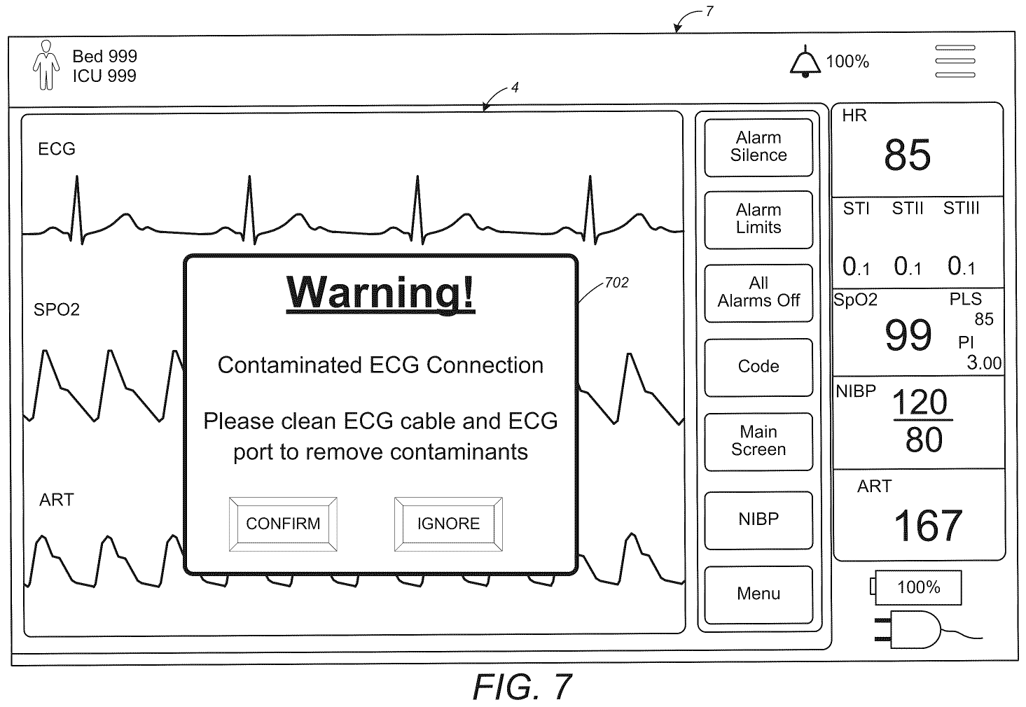

In the next step S7, the patient monitor 7 requires a user to acknowledge the alarm (as further illustrated in FIG. 7). In one embodiment, the patient monitor 7 may not allow the input port to operate until the alarm is acknowledged. Alternatively, the patient monitor 7 may simply display a temporary warning message in order to alert users of a potential problem, but will not otherwise hinder the operation of the patient monitor 7. In another embodiment, the patient monitor 7 may be prevented from operating entirely so as to prevent users from using the contaminated port and potentially clinically incorrect or corrupted information to make clinical decisions about the patient 1. In yet another embodiment, users may configure which cables and connections prevent total operation of the patient monitor 7 and which only deactivate specific ports. Thus, in some examples, a contaminated ECG port may prevent the patient monitor 7 from operating entirely. Whereas a contaminated temperature sensor would allow the patient monitor to continue to operate and only the temperature sensor input port would be prevented from operation.

Returning to the flow chart, upon alarm acknowledgment in S7, the patient monitor 7 waits until the alarm is acknowledged. Lastly, in step S8, upon alarm acknowledgement, the patient monitor 7 provides an indication that the connector should be cleaned by a clinician. Additionally, a message could then appear after the clinician indicates that the cable has been cleaned cleaned and the patient monitor 7 determines (e.g., via step S3) that the measured impendence is now above the threshold, which indicates the connection is free of contamination (e.g., step S5).

FIG. 7 is a schematic diagram illustrating one example of the alarm contamination detection acknowledgement workflow in accordance with the present disclosure. Specially, FIG. 7 illustrates an example of an alert 702 which may be presented to users in response to a contaminated cable and/or connector port. In the illustrated example, the alert identifies the contaminated cable and/or port and provide brief instructions on how to remedy the error. Selecting the “confirm” button may cause the patient monitor 7 to execute the software routine described in FIG. 7. In one embodiment, the “confirm” button may cause the software routine in FIG. 6 to start from Step S4 in order to re-check the impendence status after the cable and/or port has been cleaned and reconnected. Additionally, in some embodiments, the patient monitor 7, may be prevented from displaying patient parameter information until the patient monitor determines that the cable and/or connection port is no longer contaminated, for example, via the software routine described with respect to FIG. 6. Additionally, or alternatively, the user may be permitted to override the alert, for example, via the “ignore” acknowledgement boxes, which then enables a medical personnel to override the contamination detection algorithm and enable patient information to be displayed on the patient monitor 7.

The computer programs, which can also be referred to as programs, software, software applications, applications, components, or code, include machine instructions for a programmable processor, and can be implemented in a high-level procedural language, an object-oriented programming language, a functional programming language, a logical programming language, or an assembly language or machine language. The term computer-readable recording medium refers to any computer program product, apparatus, or device, such as a magnetic disk, optical disk, solid-state storage device, memory, and programmable logic devices (PLDs), used to provide machine instructions or data to a programmable data processor, including a computer-readable recording medium that receives machine instructions as a computer-readable signal.

By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of computer-readable media.

Use of the phrases “capable of,” “capable to,” “operable to,” or “configured to” in one or more embodiments, refers to some apparatus, logic, hardware, and/or element designed in such a way to enable use of the apparatus, logic, hardware, and/or element in a specified manner. The subject matter of the present disclosure is provided as examples of apparatus, systems, methods, and programs for performing the features described in the present disclosure. However, further features or variations are contemplated in addition to the features described above. It is contemplated that the implementation of the components and functions of the present disclosure can be done with any newly arising technology that may replace any of the above implemented technologies.

Although specific visual indications are described with reference to FIGS. 5-10 (e.g., check mark, etc.), it is contemplated by the present disclosure that almost any visual indication can be implemented that effectively conveys the status of any measurement schedule and other aspects of the GUI 50 to the user. Additionally, the above description of “selection” or “selections” as described with reference to FIGS. 5-7 (e.g., “start”, “stop”, etc.) are examples of virtual tab, buttons, icons, labels, or other selectable symbols within the GUI 50 that allow interaction between the user and the GUI 50.

Additionally, the above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

We claim:

1. A patient monitoring system, comprising:
a patient monitor including one or more processors;
one or more sensors attached to a patient and configured to measure parameters of the patient, the one or more sensors connected to the patient monitor via a cable; and
a memory on which are stored one or more software programs that are executed by the one or more processors of the patient monitor,
wherein the one or more processors are communicatively connected to the memory and configured to execute the one or more programs to:
receive the measured patient parameters;
determine an indication of a disconnected cable;
determine if a connection interface is contaminated based on a detection algorithm; and
provide an alert on a display if the detection algorithm indicates that the interface is contaminated.

2. The patient monitoring system of claim 1, wherein the patient monitor comprises a plurality of connection interfaces and the one or more processors are configured to identify which connection interface of the plurality of connection interfaces is contaminated.

3. The patient monitoring system of claim 1, wherein the detection algorithm is a lead-off detection algorithm that is executed upon a determination of a disconnected cable.

4. The patient monitoring system of claim 3, wherein the lead-off detection algorithm measures an impedance of pins of the connection interface to determine if the impedance exceeds a predefined threshold.

5. The patient monitoring system of claim 4, wherein the predefined threshold is 100 MegaOhms.

6. The patient monitoring system of claim 1, wherein the one or more processors are configured to prevent data transmission via the connection interface in response to a determination that the connection interface is contaminated.

7. The patient monitoring system of claim 6, wherein the alert includes a user-selectable interface that is configured to require a user to acknowledge the alert prior to enabling data transmission via the connection interface.

8. The patient monitoring system of claim 6, wherein the alert comprises one or more user-selectable inputs that override the prevention of the data transmission.

9. The patient monitoring system of claim 1, wherein the one or more sensors measure at least one of ECG, SpO2, NIBP, temperature, NMT, and ETCO2.

10. A method for automatic detection of contaminated connectors, the method, comprising:
attaching one or more sensors to a patient that are configured to measure parameters of the patient, the one or more sensors connected to a patient monitor via a cable;
receiving patient parameters measured by the one or more sensors attached to the patient;
determining if the cable is disconnected;
determining if a connection interface between the cable and patient monitor is contaminated based on a detection algorithm; and
displaying an alert on a display, if the detection algorithm indicates that the interface is contaminated.

11. The method of claim 10, wherein the patient monitor comprises a plurality of connection interfaces and the one or more processors are configured to identify which connection interface of the plurality of connection interfaces is contaminated.

12. The method of claim 10, further wherein the detection algorithm comprises a lead-off detection algorithm.

13. The method of claim 12, wherein the lead-off detection algorithm measures an impedance of the pins of the connection interface to determine if the impedance exceeds a predefined threshold.

14. The method of claim 13, wherein the predefined threshold is 100 MegaOhms.

15. The method of claim 10, further comprising preventing data transmission via the connection interface in response to the determination that the connection interface is contaminated.

16. The method of claim 15, wherein the alert includes a user-selectable interface that is configured to require a user to acknowledge the alert prior to enabling data transmission via the connection interface.

17. The method of claim 15, wherein the alert comprises one or more user-selectable inputs that override the prevention of the data transmission.

18. The method of claim 10, wherein the one or more sensors measure at least one of ECG, SpO2, NIBP, temperature, NMT, and ETCO2.
